(19) United States
(12) Patent Application Publication
(10) Pub. No.: US 2004/0220513 A1
(43) Pub. Date: Nov. 4, 2004

(54) Low level light therapy for the enhancement of hepatic functioning

(76) Inventor: Jackson Streeter, Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/793,174

(22) Filed: Mar. 4, 2004

Related U.S. Application Data
(60) Provisional application No. 60/452,048, filed on Mar. 4, 2003.

(51) Int. Cl. A61N 1/30

(52) U.S. Cl. 604/20; 607/88

(57) ABSTRACT

Therapeutic methods for the enhancement of hepatic functioning are described, the methods including delivering a heptatic enhancement effective amount of light energy having a wavelength in the visible to near-infrared wavelength range to a target area of the liver. A heptatic enhancement effective amount of light energy is a selected or predetermined power density (mW/cm²) at the level of the liver tissue being treated, and may be determined by determining a surface power density of the light energy sufficient to deliver the selected power density of light energy to the target liver tissue taking into account factors that attenuate the light energy as it travels from the skin surface to the tissue being treated. The disclosed methods have utility in treatment of liver disease as well as in enhancing the functioning of a normal liver.

### RELATED APPLICATION INFORMATION

This application claims priority under 35 U.S.C. § 119(e) to U.S. Provisional Application Ser. No. 60/452,048 filed Mar. 4, 2003.

### BACKGROUND OF THE INVENTION

1. Field of the Invention

The present invention relates in general to therapeutic methods for the enhancement of hepatic functioning, and more particularly to novel methods for enhancing the hepatic functioning, including the production of biologically important proteins, using low level light therapy. Such methods have utility in treatment of liver disease as well as in enhancing the functioning of a normal liver.

2. Description of the Related Art

The liver is one of the largest organs in the body, and plays an important role in a wide variety of functions including digestion, metabolism, detoxification, and elimination of waste products. The liver also produces several important proteins, including albumin which is the major plasma protein (approximately 60 percent of the total), and is responsible for much of the plasma colloidal osmotic pressure and serves as a transport protein carrying large organic anions, such as fatty acids, bilirubin and many drugs and also certain hormones, such as cortisol and thyroxine, when their specific binding globulins are saturated. Low serum levels occur in protein malnutrition, active inflammation and serious hepatic and renal disease.

High energy laser radiation is now well accepted as a surgical tool for cutting, cauterizing, and ablating biological tissue. High energy lasers are now routinely used for vaporizing superficial skin lesions and, and to make deep cuts. For a laser to be suitable for use as a surgical laser, it must provide laser energy at a power sufficient to heat tissue to temperatures over 50° C. Power outputs for surgical lasers vary from 1-5 W for vaporizing superficial tissue, to about 100 W for deep cutting.

In contrast, low level laser therapy involves therapeutic administration of laser energy to a patient at vastly lower power outputs than those used in high energy laser applications, resulting in desirable biostimulatory effects while leaving tissue undamaged. Low level laser therapy has been described for treating pain, including headache and muscle pain, and inflammation.

### SUMMARY OF THE INVENTION

In accordance with a preferred embodiment, there is provided a method for the enhancement of hepatic functioning in a subject, said method comprising delivering a hepatic enhancement effective amount of light energy having a wavelength in the visible to near-infrared wavelength range to a target area of the liver of the patient, wherein delivering the hepatic enhancement effective amount of light energy comprises delivering a specified power density of light energy to the area of the liver.

Additional preferred embodiments of the foregoing methods may include one or more of the following: the selected power density is a power density selected from the range of about 0.01 mW/cm² to about 150 mW/cm²; the light energy has a wavelength of about 780 nm to about 840 nm; and the light is delivered in pulses at a frequency of about 1 Hz to about 1 kHz.

Preferred methods may further encompass selecting a dosage and power of the light energy sufficient to deliver the predetermined power density of light energy to the liver by selecting the dosage and power of the light sufficient for the light energy to penetrate any body tissue, for example a thickness of skin and other bodily tissue such as fat and muscle that is interposed between the liver and the skin surface adjacent the liver and/or sufficient for the light energy to traverse the distance between the liver and the skin surface adjacent the liver.

### BRIEF DESCRIPTION OF THE DRAWINGS

FIG. 1 is a perspective view of a first embodiment of a light therapy device; and

FIG. 2 is a block diagram of a control circuit for the light therapy device, according to one embodiment of the invention.

FIG. 3 illustrates the results of an experiment in which primary rat hepatocytes were established in a conventional collagen sandwich culture, stabilized for 7 days, then treated daily with 830 nm light from a Gallium-Aluminum-Arsenide diode laser at a constant power density of 50 mW/cm² and compared to controls.

### DETAILED DESCRIPTION OF THE PREFERRED EMBODIMENT

The lower level light therapy methods for the enhancement of hepatic function described herein are practiced and described using, for example, a low level light therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference together with the references contained therein.

A suitable apparatus for the methods disclosed herein is a low-level light apparatus including a handheld probe for delivering the light energy. The probe includes a light source of light energy having a wavelength in the visible to near-infrared wavelength range, i.e. from about 630 nm to about 904 nm. In one embodiment, the probe includes a single laser diode that provides about 25 mW to about 500 mW of total power output, or multiple laser diodes that together are capable of providing at least about 25 mW to about 500 mW of total power output. In other embodiments, the power provided may be more or less than these stated values. The actual power output is preferably variable using a control unit electronically coupled to the probe, so that the power of the light energy emitted can be adjusted in accordance with required power density calculations as described below. In one embodiment, the diodes used are continuously emitting GaAIAs laser diodes having a wavelength of about 830 mm.

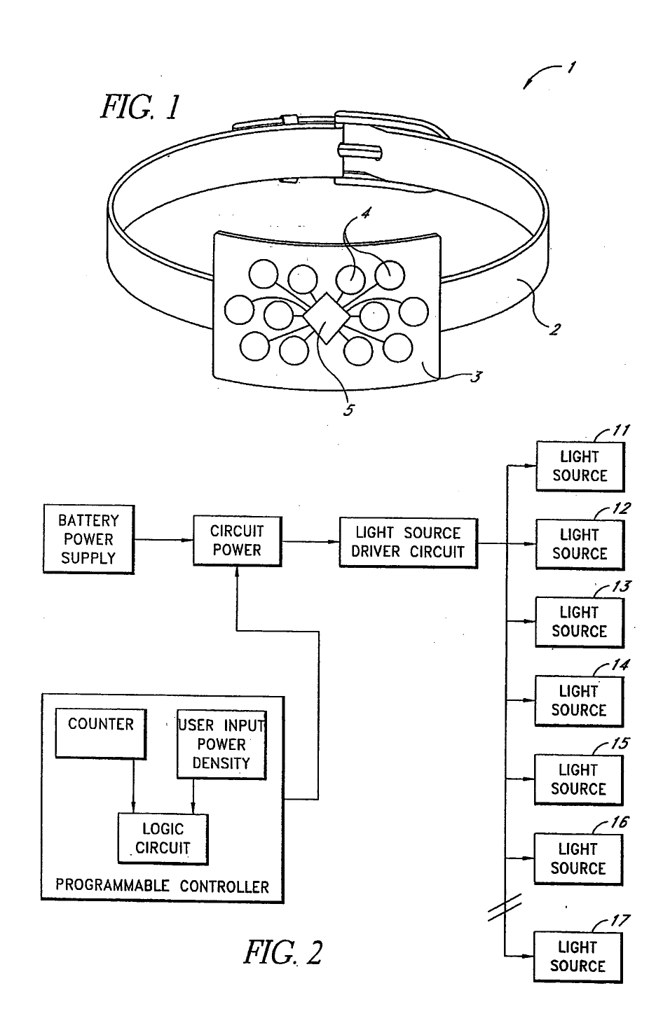

Another suitable light therapy apparatus is that illustrated in FIG. 1. This apparatus is especially preferred for methods in which the light energy is delivered through the skin. The illustrated device 1 includes a flexible strap 2 with a securing means, the strap adapted for securing the device over an area of the subject's body, one or more light energy sources 4 disposed on the strap 2 or on a plate or enlarged portion of the strap 3, capable of emitting light energy having a wavelength in the visible to near-infrared wavelength range, a power supply operatively coupled to the light source or sources, and a programmable controller 5 operatively coupled to the light source or sources and to the power supply. Based on the surprising discovery that control or selection of power density of light energy is an important factor in determining the efficacy of light energy therapy, the programmable controller is configured to select a predetermined surface power density of the light energy sufficient to deliver a predetermined subsurface power density to a body tissue to be treated beneath the skin surface of the area of the subject's body over which the device is secured.

The light energy source or sources are capable of emitting the light energy at a power sufficient to achieve the predetermined subsurface power density selected by the programmable controller. Suitable parameters for the light that is delivered is discussed elsewhere infra.

The strap is preferably fabricated from an elastomeric material to which is secured any suitable securing means, such as mating Velcro strips, snaps, hooks, buttons, ties, or the like. Alternatively, the strap is a loop of elastomeric material sized appropriately to fit snugly around the chest. The precise configuration of the strap is subject only to the limitation that the strap is capable of maintaining the light energy sources in a select position relative to the particular area of the body or tissue being treated. In an alternative embodiment, a strap is not used and instead the light source or sources are incorporated into or attachable onto a piece of fabric which fits securely over the target body portion thereby holding the light source or sources in proximity to the patient's body for treatment. The fabric used is preferably a stretchable fabric or mesh comprising materials such as Lycra or nylon. The light source or sources are preferably removably attached to the fabric so that they may be placed in the position needed for treatment.

In the exemplary embodiment illustrated in FIG. 1, a light therapy device includes a flexible strap and securing means such as mating Velcro strips configured to secure the device around the body of the subject. The light source or sources are disposed on the strap, and in one embodiment are enclosed in a housing secured to the strap. Alternatively, the light source or sources are embedded in a layer of flexible plastic or fabric that is secured to the strap. In any case, the light sources are preferably secured to the strap so that when the strap is positioned around a body part of the patient, the light sources are positioned so that light energy emitted by the light sources is directed toward the skin surface over which the device is secured. Various strap configurations and spatial distributions of the light energy sources are contemplated so that the device can be adapted to treat different tissues in different areas of the body. Furthermore, the device may be provided without a strap and placed over the area of treatment either with or without additional securement.

FIG. 2 is a block diagram of a control circuit according to one embodiment of the light therapy device. The programmable controller is configured to select a predetermined surface power density of the light energy sufficient to deliver a predetermined subsurface power density, preferably about 0.01 mW/cm² to about 150 mW/cm², including about 10 mW/cm² to about 100 mW/cm² to the target area. The actual total power output if the light energy sources is variable using the programmable controller so that the power of the light energy emitted can be adjusted in accordance with required surface power energy calculations as described below.

The methods described herein are based in part on the surprising finding that delivering low level light energy within a select range of power density (i.e. light intensity or power per unit area, in mW/cm²) appears to be an important factor for producing therapeutically beneficial effects with low level light energy as applied to liver tissue. Without being bound by theory, it is believed that independently of the power and dosage of the light energy used, light energy delivered within the specified range of power densities provides a biostimulative effect on the intracellular environment, such that proper function is returned to previously non-functioning or poorly functioning cells or that healthy cells function at an improved level.

The term “hepatic enhancement” refers to a therapeutic strategy for improving the functioning of hepatic tissues, whether in an healthy or diseased state. Diseased states include those associated with hepatitis or other viral diseases, cirhhosis, autoimmune disorders, trauma, and other conditions which functioning of the liver.

The term “hepatic enhancement effective” as used herein refers to a characteristic of an amount of light energy, wherein the amount is a power density of the light energy measured in mW/cm². The amount of light energy achieves the goal of enhancing hepatic function.

In preferred embodiments, treatment parameters include the following. Preferred power densities of light at the level of the target cells are at least about 10 mW/cm² to about 150 mW/cm², including about 15, 20, 30, 40, 50, 60, 70, 80, 90, 100, 110, 120, 130, and 140 mW/cm². In some embodiments, higher power densities can be used. To attain subsurface power densities within this preferred range in in vivo methods, one must take into account attenuation of the energy as it travels through body tissue and fluids from the surface to the target tissue, such that surface power densities of from about 25 mW/cm² to about 500 mW/cm² will typically be used to achieve such power densities at the level of the target tissue, but higher values, such about 1000 mW/cm² or more, may also be used. To achieve desired power densities, preferred light energy sources, or light energy sources in combination, are capable of emitting light energy having a total power output of at least about 0.01 mW to about 500 mW, including about 0.05, 0.1, 0.5, 1, 2, 5, 10, 15, 20, 30, 50, 75, 100, 150, 200, 250, 300, and 400 mW, but may also be up to as high as about 1000 mW or below 1 mW. Preferably the light energy used for treatment has a wavelength in the visible to near-infrared wavelength range, i.e., from about 630 to about 904 nm, preferably about 780 nm to about 840 nm, including about 790, 800, 810, 820, and 830 nm.

In preferred embodiments, the light source used in the light therapy is a coherent source (i.e. a laser), and/or the light is substantially monochromatic (i.e. one wavelength or a very narrow band of wavelengths).

In preferred embodiments, the treatment proceeds continuously for a period of about 30 seconds to about 4 hours, including about 10 minutes, 20 min., 30 min., 45 min., 1 hour, 2 hrs., and 3 hrs. Treatment times outside of these ranges are also within the scope of the invention, and may be performed as deemed necessary for effective treatment. The treatment may be terminated after one treatment period, or the treatment may be repeated one or more times, with anywhere from a few hours to a few days passing between treatments. The length of treatment time and frequency of treatment periods can be varied as needed to achieve the desired result.

During the treatment, the light energy may be continuously provided, or it may be pulsed. If the light is pulsed, the pulses are preferably at least about 10 ns long, including about 100 ns, 1 ms, 10 ms, and 100 ms, and occur at a frequency of up to about 1 kHz, including about 1 Hz, 10 Hz, 50 Hz, 100 Hz, 250 Hz, 500 Hz, and 750 Hz.

Generally, light energy suitable for practicing the methods includes light energy in the visible to near-infrared wavelength range, i.e. wavelengths in the range of about 630 nm to about 904 nm. In an exemplary embodiment, the light energy has a wavelength of about 830 nm, as delivered with laser apparatus including GaAlAs diodes as the laser energy source.

Thus, a method for the enhancement of hepatic functioning in a subject in need of such treatment involves delivering a hepatic enhancement effective amount of light energy having a wavelength in the visible to near-infrared wavelength range to a target area of the liver of the subject. The target area may be a portion of the liver or it may be the entire liver such that the treatment may be carried out by treating smaller sections of the liver in sequence. In preferred embodiments, delivering the hepatic enhancement effective amount of light energy includes selecting a surface power density of the light energy sufficient to deliver a predetermined power density of light energy to the target area of the liver. The power density to be delivered to the tissue is selected to be at least about 0.01 mW/cm², preferably about 10 mW/cm² or more. In one embodiment, the selected or predetermined power density to be delivered to the tissue is selected from the range of about 0.01 mW/cm² to about 150 mW/cm², including about 1 mW/cm² to about 20 mW/cm².

To deliver the desired power density at the level of the liver tissue, a relatively greater surface power density of the light energy is needed, and is calculated taking into account attenuation of the light energy as it travels from the skin surface through various tissues including skin, bone and fat tissue. Factors known to affect penetration and to be taken into account in the calculation include skin pigmentation, and the location of the affected or target area, particularly the depth of the area to be treated relative to the surface. The higher the level of skin pigmentation, the higher the required surface power density to deliver a predetermined power density of light energy to a subsurface site in the liver.

To treat a patient, the light source is placed in contact with or immediately adjacent to a region of skin, for example on the back or side of the right side of the patient, adjacent to the target section(s) of the liver. The target section may be determined using medical techniques including, but not limited to, CT and MRI. The power density calculation to determine how much power needs to be delivered at the surface preferably takes into account factors including skin coloration, distance to the target site in the liver, etc. that affect penetration and thus power density at the target site, and the power used and the surface area treated are adjusted accordingly.

The precise power density selected for treating a patient depends on a number of factors, including the specific wavelength of light selected, the extent of the target tissue, the clinical condition of the subject, and the like. Similarly, it should be understood that the power density of light energy to be delivered to the target area may be adjusted to be combined with any other therapeutic agent or agents. The selected power density will again depend on a number of factors, as noted above, also including the particular therapeutic agent(s) employed.

### EXAMPLE 1

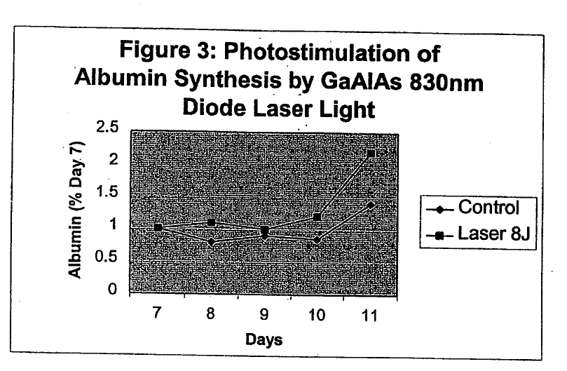

An in vitro experiment was conducted to demonstrate some effects of light therapy according to a preferred embodiment on rat hepatocytes. Primary rat hepatocytes were established in a conventional collagen sandwich culture and then stabilized for 7 days. The cells were maintained in triplicate 35 mm cultures, containing 500,000 primary hepatocytes in 1.5 ml volumes of culture media that was changed daily. The cultured cells were then treated daily with 830 nm light from a Gallium-Aluminum-Arsenide diode laser at a constant power density of 50 mW/cm² for up to 160 seconds through a lighttable into which the laser was mounted, to achieve doses of up to 8 J/cm². The albumin production levels of the treated cells were compared to that of non-lased controls. The data are presented in FIG. 3.

These experiments show that albumin synthesis of cells treated with light therapy is increased up to 115%. These results show that 830 nm light has a potent effect on hepatocyte function that is observable after just 72 hours—a time frame typically required in cell culture systems to induce stable repatterning of nuclear gene expression associated with a new cellular differentiation state (Kluge et al., 1974; Roman et al., 1992). In this experiment, albumin synthesis in untreated control cultures increased less than 45%.

Cells from this same series of experiments were also examined for cell viability using the Calcein AM/Ethidium Homodimer-1 system (Molecular Probes, Eugene, Oreg.) and intracellular lipid accumulation by phase contrast photomicroscopy on Day 6 of treatment (Day 12 in the number scheme used in FIG. 8). Control cells accumulated more microvesicular lipid (visible as highly refractile cytoplasmic accumulations) and were also found to contain non-refractile cells, which are dead or dying cells. Cells that received light treatment had much reduced lipid and superior viability compared to untreated control cells (which showed more than 20 red-stained nuclei per low power field).

These results are novel and significant in that they show the positive effects of laser irradiation on cellular functioning and viability in in vitro, primary rat hepatocyte cell cultures.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for the enhancement of hepatic functioning in a subject, said method comprising delivering a hepatic enhancement effective amount of light energy having a wavelength in the visible to near-infrared wavelength range to a target area of the liver of the patient, wherein delivering the hepatic enhancement effective amount of light energy comprises delivering a specified power density of light energy to the target area of the liver.

2. A method in accordance with claim 1 wherein the predetermined power density is a power density selected from the range of about 0.01 mW/cm² to about 150 mW/cm².

3. A method in accordance with claim 2 wherein the predetermined power density is selected from the range of about 1 mW/cm² to about 20 mW/cm².

4. A method in accordance with claim 1 wherein the light energy has a wavelength of about 630 nm to about 904 nm.

5. A method in accordance with claim 4 wherein the light energy has a wavelength of about 830 nm.

6. A method in accordance with claim 1 wherein delivering a hepatic enhancement effective amount of light energy to a target area of the liver of the subject comprises placing a light source in contact with a region of skin adjacent the area of the liver.

7. A method in accordance with claim 1 wherein delivering a hepatic enhancement effective amount of light energy to a target area of the liver of the subject comprises determining a surface power density of the light energy sufficient to deliver the power density of light energy to the target area of the liver.

8. A method in accordance with claim 7 wherein determining a surface power density comprises determining the surface power density of the light energy sufficient for the light energy to traverse the distance between the skin surface and the target area of the liver.

9. A method in accordance with claim 8 wherein determining the surface power density further comprises determining the surface power density sufficient to penetrate body tissue between the skin surface and the target area of the liver.

10. A method in accordance with claim 1, wherein the light is delivered in pulses at a frequency of about 1 Hz to about 1 kHz.
